(19) United States
(12) Patent Application Publication
(10) Pub. No.: US 2004/0214750 A1
(43) Pub. Date: Oct. 28, 2004

(54) Medicaments for healing skin conditions in humans

(76) Inventor: Izolda M. Georgiades, Houston, TX (US)

Correspondence Address: Kurt S. Myers, 7634 Braesdale, Houston, TX, 77071, US

(21) Appl. No.: 10/832,690

(22) Filed: Apr. 27, 2004

Related U.S. Application Data
(60) Provisional application No. 60/465,944, filed on Apr. 28, 2003.

(51) Int. Cl. A61K 38/40

(52) U.S. Cl. 514/6; 424/70.14

(57) ABSTRACT

The present invention is directed to a novel progression of topical and oral administration or use of lactoferrin or lactoferrin and other biological ingredients to prevent and treat dermatological conditions or disorders in humans. More specifically, the present invention is directed to the administration of lactoferrin in a topical material such as an ointment or cream to the skin of humans and also including a oral administration, together the topical and oral administration has a synergetic effect in the treatment of the skin. The progression of medicaments of the present invention are: 1) lactoferrin; 2) lactoferrin and conjugated linoleic acids; 3) lactoferrin, a plant or animal derived lipid and oblepicha; 4) lactoferrin, a plant or animal derived lipid, oblepicha, and an omega fatty acid; and 5) lactoferrin, a plant or animal derived lipid, oblepicha, an omega fatty acid and a whey protein.

### RELATED APPLICATION

This application is based on provisional application 60/465,944 filed Apr. 28, 2003.

### FIELD OF THE INVENTION

The present invention is directed to a novel administration or use of lactoferrin or lactoferrin and a progression of other specific biological ingredients to prevent and treat dermatological conditions or disorders in humans. More specifically, the present invention is directed to the administration of lactoferrin in a topical material such as an ointment or cream to the skin of humans and also including a oral administration, together the topical and oral administration has a synergetic effect in the treatment of the skin.

### BACKGROUND OF THE INVENTION

The principal functions of the skin include protection, excretion, secretion, absorption, thermoregulation, sensory perception and regulation of immunological processes. These functions are all affected by the structural changes in the skin with ageing. The physiological changes associated with ageing (these reductions) include impairment of the barrier function, decreased turnover of epidermal cells, reduced numbers of keratinocytes and fibroblasts, and a reduced vascular network. Certain environmental factors, particularly exposure to sun, accelerate the ageing of skin and are important in cutaneous carcinogenesis. Regarding the outdoor behavior of the contemporary population modern sunscreens should provide high and broad-spectrum ultraviolet protection in the ultraviolet B as well as in the ultraviolet A range and should be photochemically stable for ultraviolet doses, which can be expected in solar radiation. Prevention of skin senescence relies in part on sun protection, tobacco avoidance and limitation of sustained mechanical stress to the skin. Free radical scavengers in adequate combinations are major contributors to slow down the ageing process. Vitamins E and C, as well as selenium and some plant extracts showed some positive effects in protection of skin against environmental insults.

U.S. Pat. No. 4,342,747 discloses colostrums-based compositions for external ophthalmic use as a therapeutic agent and/or as a tear substitute.

U.S. Pat. No. 5,698,185 discloses a whitening cosmetic preparation containing angiogenin as an effective component, the angiogenin may be derived from milk, especially bovine milk.

U.S. Pat. No. 5,750,149 discloses pharmaceutical and/or dermocosmetic compositions containing equine colostrums, optionally in admixture with milk.

U.S. Pat. No. 6,187,297 discloses a composition that includes Sea Buckthorn Oil as a cosmetic product, a pharmaceutical or nutraceutical product or a food product. The food product can be used in combination with the cosmetic applications of the present invention.

U.S. Pat. No. 6,221,918 discloses combinations of carotenoids for use as a dietary supplement or topical ointment.

2001/0000466 discloses a composition that includes Sea Buckthorn Oil as a cosmetic product, a pharmaceutical or nutraceutical product or a food product.

U.S. Pat. No. 6,258,383 discloses a dietary supplement comprising colostrum, lactoferrin, and with modified citrus pectin as an optional component, administered in ‘mucosal delivery format’: a dosage form that promotes effective absorption through the lining of the oral cavity.

2001/0009681 discloses a dietary supplement comprising colostrum, lactoferrin, and with modified citrus pectin as an optional component, administered in ‘mucosal delivery format’: a dosage form that promotes effective absorption through the lining of the oral cavity.

U.S. Pat. No. 6,319,522 discloses pharmaceutical or veterinary compositions for the treatment of surface wounds; pharmaceutical or veterinary compositions for the treatment of gastrointestinal injuries, diseases or ulcers; methods of treating surface wounds in animals, including humans; and methods for the treatment of gastrointestinal injuries, diseases or ulcers which compositions and methods include compositions of milk product extracts including growth factors with basic to approximately neutral isoelectric points.

U.S. Pat. No. 6,410,058 discloses a dietary supplement comprising colostrum, lactoferrin, and with modified citrus pectin as an optional component, administered in ‘mucosal delivery format’: a dosage form that promotes effective absorption through the lining of the oral cavity.

2002/0001625 discloses pharmaceutical or veterinary compositions for the treatment of surface wounds; pharmaceutical or veterinary compositions for the treatment of gastrointestinal injuries, diseases or ulcers; methods of treating surface wounds in animals, including humans; and methods for the treatment of gastrointestinal injuries, diseases or ulcers which compositions and methods include compositions of milk product extracts including growth factors with basic to approximately neutral isoelectric points.

2002/0054917 discloses dietary supplement compositions containing beta glucan and lactoferrin, formulated as a liquid, tablet, lozenge, gum or other acceptable form.

U.S. Pat. No. 6,447,808 discloses pharmaceutical or veterinary compositions for the treatment of surface wounds; pharmaceutical or veterinary compositions for the treatment of gastrointestinal injuries, diseases or ulcers; methods of treating surface wounds in animals, including humans; and methods for the treatment of gastrointestinal injuries, diseases or ulcers which compositions and methods include compositions of milk product extracts including growth factors with basic to approximately neutral isoelectric points.

U.S. Pat. No. 6,475,511 discloses a dietary supplement comprising colostrum, lactoferrin, and with modified citrus pectin as an optional component, administered in ‘mucosal delivery format’: a dosage form that promotes effective absorption through the lining of the oral cavity.

U.S. Pat. No. 6,479,627 discloses an aqueous preparation of lactoferrin where stability of lactoferrin in an aqueous solution is improved and also to a method of stabilizing lactoferrin in an aqueous solution.

### SUMMARY OF THE INVENTION

The present invention is directed to a novel progression of topical and oral administration or use of lactoferrin to prevent and treat dermatological conditions or disorders in humans. More specifically, the present invention is directed to the administration of lactoferrin in a topical material such as an ointment or cream to the skin of humans and also including a oral administration, together the topical and oral administration has a synergetic effect in the treatment of the skin. The progression of medicaments of the present invention involve the addition to lactoferrin of other biologically active compounds as follows: 1) lactoferrin; 2) lactoferrin and conjugated linoleic acids (CLA); 3) lactoferrin, an animal or plant-derived lipid and oblepicha; 4) lactoferrin, an animal or plant-derived lipid, oblepicha, and an omega fatty acid; and 5) lactoferrin, an animal or plant-derived lipid, oblepicha, an omega fatty acid and a whey protein.

### BRIEF DESCRIPTION OF THE PRESENT INVENTION

The present invention is directed to active ingredients containing isolated lactoferrin, a protein isolated from milk or colostrums (mother's first milk). More specifically, the present invention is directed to a progression of medicaments containing lactoferrin and other compounds for the protective or therapeutic application to skin disorders in humans.

A specific aspect of the present invention is that the medicaments are administered both topically and internally. This regimen of administration has resulted in much quicker (meaning shorter time for positive results to be obtained) treatment of the patient. The progression of compounds added to the lactoferrin provide a series of products that are especially suited for the phases that skins have over the period of life as the person ages. It is a specific aspect of the present invention and a finding made according to the present invention that one composition where lactoferrin is the only active ingredient does not work on all skins.

During a person's life span, changes in skin are associated with the degeneration of cells and tissues which can result in ageing or diseases, such as bacterial, fungal or yeast infections, cancer, cardiovascular failure, neuro-generative disorders and oxidative distress. Skin ageing is the global result of the influence of numerous factors. However, skin is subject to more than aging but also to injury such as burns or broken bones. It is clear therefore that a baby's skin and an elderly person's skin is not the same.

Skin is a major defense system organ which protects our body against the microbial infections, regulates body temperature as well as controls the oxygen—mineral—water exchange ratio. Skin provides a very efficient excretion mechanism for many metabolic end products, including inorganic elements such as iron, phosphate, ammonium, sodium, potassium to name a few. In general, products secreted by skin with sweat are toxic and should be neutralized or removed from the skin immediately. Perhaps the most dangerous factor secreted with sweat is iron. Daily hygiene of skin is the most important factor in promoting a healthy lifestyle.

The principal functions of the skin include protection, excretion, secretion, absorption, thermoregulation, sensory perception and regulation of immunological processes. These functions are all affected by the structural changes in the skin with ageing. The physiological changes associated with these ageing structural changes include impairment of the barrier function, decreased turnover of epidermal cells, reduced numbers of keratinocytes and fibroblasts, and a reduced vascular network. Certain environmental factors, particularly exposure to sun, induce overproduction of free radicals that accelerate the ageing of skin and cause cutaneous carcinogenesis.

Lactoferrin is a well documented protein. The lactoferrin used in the present invention is exogenous or isolated lactoferrin and may be lactoferrin recovered from human, bovine, sheep or porcine milk. A preferred lactoferrin is that recovered from bovine milk which is commercially available.

The products of the present invention are produced by mixing lactoferrin, alone or with specific biologically active ingredients that provide specific added effect to the prevention or alleviation of the common conditions of human skin. These ingredients are pharmaceutically active materials that produce a product that is administered to the skin as a cream, a gel, an aqueous solution, a spray or a trans-dermal patch, to name a few or administered orally as a capsule or tablet. Pharmaceutically active ingredients include but are not limited to:

conjugated linoleic acids (CLA) CLA has the structure: cis-9, trans-11 (C18:2), with the double bonds separated by only one single bond. The preferred CLA is a natural product obtained from sunflower seeds.

a plant-derived lipid extract, such as a lipid from leaves, fruits or seeds of commercially available plants such as raspberries, blackberries or Hippohae rhamnoides L. The preferable plant extract is obtained from the fruits of Hippohae rhamnoides L., also called oblepicha

omega fatty acids

whey peptides

The present invention is directed to the progression of use of these ingredients to show that each has a specific function and that favorable results are obtained when the specific ingredients are used in a specific formulation for specific skin conditions.

To illustrate the present invention a topical and an oral medicament containing lactoferrin is produced:

### EXAMPLE 1

Anti Acne Formula:

Topical Product 1: each of the following ingredients is placed into a commercial mixer:

1-5 g. of bovine lactoferrin;

0.2% Germall 115;

4% grape-stone oil;

3% Myristic isopropyl;

3%, glycol extract from bluebottle;

0.2% Phenova;

3% glycerin;

3% Sepigel 305,

82.6-77.6% of water; per 100 g of final mixture. If necessary the materials are passed through a #10-12 mesh-screen to remove aggregates. Each of the procedures should be performed with precautions against exposure to the vapors that are formed, and particularly against their inhalation. After 30 minutes of thorough mixing the formula is placed into glass jars.

This cream is particularly effective in various skin bacterial manifestations such as acne vulgaris or acne rosecea. It is also very good for young skin not yet damaged by oxidative processes induced by sweat or pathological conditions such as irradiation or iron damaged skin, a result of the Fenton reaction.

The cream was applied to 39 volunteers twice a day, morning and evening. After 10 weeks of treatment most of the participants in this study showed significant improvement. This is summarized below:

Treatment.

| Variable | Before | After | Improvement |
|---|---|---|---|
| Nest eggs | 30 | 8 | 73.4% |
| Oily skin | 16 | 3 | 92.3% |
| Acne | 31 | 4 | 87.1% |

As may be seen significant improvement of the skin condition following 10 weeks of anti acne formula application was noted. Reduction of pore size was one of additional effect of anti acne cream application.

Oral Product 1: each of the following ingredients is placed into a commercial mixer:

1 g. of bovine lactoferrin;

sunflower oil to make a liquid or incorporate liquid in common fillers to make a capsule.

Using an administration of Topical Product 1 twice daily and oral administration of the Oral Product 1 twice daily, a significant improvement of skin condition was noted in ten days.

### First Progression

The first progression of the present invention includes lactoferrin and conjugated linoleic acids (CLAs). that improve the medicaments of the present invention.

### EXAMPLE 2

Product 2: each of the following ingredients is placed into a commercial mixer:

0.5-2% of lactoferrin;

0.5-2% of CLA;

2% Cremophore A 25;

2% Cremophore A-6;

1-2% Cutina GMS;

0.5-1.5% Beewax;

0.5-6% Grape-stone oil;

2-6% Coconut oil;

2-3% Myristic isopropyl;

0.2-3% Germall 115;

0.2% Phenova;

0.2-3% propylene glycol;

0.3-3% Carbopol 940;

0.3-0.4% triethyl-amine;

87.8-63.9% water; per 100 g of final mixture. If necessary the materials are passed through a #10-12 mesh-screen to remove aggregates. Each of the procedures should be performed with precautions against exposure to the vapors that are formed, and particularly against their inhalation. After 30 minutes of thorough mixing the formula is placed into glass jars. This formula is specially designed for mature skin, having tendency to loose its moisture and formulation of wrinkles. This formula was administered to subject B, an adult female (34 years) with a general dryness of the facial skin. A good looking appearance become visible after one week of treatment with a twice daily protocol.

This formula is specially designed for skin having a tendency for bacterial infections and wrinkles. It protected skin against free radical damages causing wrinkles. This product was used in cases of skin subjected to different procedures of hair removal. Since these procedures always left open scars, skin bacteria infected the skin lesion, frequently causing inflammation. In four separate occasions immediately after the removal process, application of the above cream prevented inflammation formation. In addition, the above cream was used to prevent wrinkle formation. Four young volunteers spending significant time in out-door activities reported that after 10 weeks application of the above cream their skin remained smooth and radiant and without wrinkles

Oral Product 2: each of the following ingredients is placed into a commercial mixer:

0.5-2 g. of bovine lactoferrin;

0.5-2 g of CLA

sunflower oil to make a liquid or incorporate liquid in common fillers to make a capsule.

Using an administration of Topical Product 2 twice daily and oral administration of the Oral Product 2 twice daily, a significant improvement of skin condition was noted in five to fifteen days.

### Second Progression

The second progression of the present invention includes lactoferrin, lipids, such as conjugated linoleic acids (CLAs) and oblepicha that improves the medicaments of the present invention.

Oblepicha, also known as Sea Buckthorn or the Siberian Orangeberry, comes from the Altay Region of Siberia. It is highly unusual for it's rare combination of valued vitamins, essential unsaturated fatty acids, flavonoids and minerals

This formula is particularly good for skin having a tendency for bacterial, yeast or fungus infection. The CLA's have the ability to increase cellular immunity, increasing an important element in anti-microorganism defenses. Plant mixtures having flavonoids and various vitamins, secure correct skin metabolism, sustain shiny skin and radiant appearance.

### EXAMPLE 3

Maintenance Formula for Mature Skin.

Product 3: Each of the following ingredients is placed into a commercial mixer:

1-5% of lactoferrin isolated from milk;

0.5-5%; CLA

1-10% oil extract from Sea buckthorn (Hippophe rhamnoides) plant mixture containing Vitamins and Provitamin A (Flavonoids);

2% cetyl alcohol;

3% vaselin;

2% Soya oil;

3% grape-stone oil;

3% lanolin;

2% myristic isopropyl;

0.2% Germall 115;

0.2% Phenova;

3% glycerin;

3% Sepigel 305 and

76.1-58.6% water per 100 g of final mixture. If necessary the materials are passed through a #10-12 mesh-screen to remove aggregates. Each of the procedures should be mixed with precautions against exposure to the vapors that are formed, and particularly against their inhalation. After 30 minutes after thorough mixing the formula is placed into glass jars.

This skin maintenance formula was used by twenty one people. After a few weeks of application (3-10) all participants were aware of its antibacterial potency and inhibition of stages leading to deep wrinkle formation, decrease in diameter of enlarged pores, decrease in visibility of scars and changes in skin color. Skin became smooth, more radiant and brighter.

Oral Product 3: each of the following ingredients is placed into a commercial mixer:

1-5 g. of bovine lactoferrin;

0.5-5 g of CLA

1-10 g. of Sea buckthorn

sunflower oil to make a liquid or incorporate liquid in common fillers to make a capsule.

Using an administration of Topical Product 3 twice daily and oral administration of the Oral Product 3 twice daily, a significant improvement of skin condition was noted.

### Third Progression

The third progression of the present invention includes lactoferrin, lipids, such as conjugated linoleic acids (CLAs), oblepicha, and an omega fatty acid that improves the medicaments of the present invention.

This formula is specially designed for mature skin, having a tendency to loose its moisture and formation of wrinkles. It contains strong antioxidant complex modulating activity.

### EXAMPLE 4

Nourishing Formula.

Product 4: This product contains the following ingredients:

1-5% lactoferrin isolated from milk;

0.5-5% of CLA or an animal or plant-derived lipid;

1-5% of plant-derived oil extracts from fruits (Hippophe rhamnoides);

1-5% omega fatty acids;

2% Cremophore A 25;

2% Cremophore A-6;

1.5% Cutina GMS;

2% cetyl alcohol;

4% vaselin;

3% grape-stone oil;

4% Soya oil;

2% myristic isopropyl;

1.5% Germall 115;

0.2% Phenova;

0.4% Carbopol 940;

0.5% triethyl-amine;

73.4-56.9% water; per 100 g of final mixture. If necessary the materials are passed through a #10-12 mesh-screen to remove aggregates. Each of the procedures should be performed with precautions against exposure to the vapors that are formed, and particularly against their inhalation. After 30 minutes of thorough mixing the formula is placed into glass jars.

This cream was tested on twenty six volunteers for its protective and regenerating property. All who tested had dry skin, with different types of wrinkles. All were above 55 years of age, the color of their skin was bright, a diverse number of blood vessels showed and each had decreased skin elasticity. The raised lines in the skin caused skin aching, induced inflammatory reaction and burns. In contrast the skin when treated with the above cream became well moisturized, smooth and glossy. Wrinkles become more superficial and less visible.

Oral Product 4: each of the following ingredients is placed into a commercial mixer:

1-5 g. of bovine lactoferrin;

0.5-5 g of CLA

1-5 g. of Sea buckthorn

1-5 g. omega fatty acids

sunflower oil to make a liquid or incorporate liquid in common fillers to make a capsule.

Using an administration of Topical Product 4 twice daily and oral administration of the Oral Product 4 twice daily, a significant improvement of skin condition was noted.

### Fourth Progression

The fourth progression of the present invention includes lactoferrin, lipids, such as conjugated linoleic acids (CLAs), oblepicha, an omega fatty acid and whey proteins that improves the medicaments of the present invention.

This formula is particularly good for mature skin seriously damaged by accidental exposure to heat, corrosive agent or high energy irradiation.

### EXAMPLE 5

Rejuvenating Formula

Product 5: A rejuvenating formula is prepared from the following ingredients:

1-5% of lactoferrin isolated from milk;

0.5-5% of plant CLA;

1-5% oil extract from Hippochae rhamnoides,

1-5% omega fatty acid;

1-5% whey proteins;

3% vaselin;

2% Soya oil;

3% grape-stone oil;

3% lanolin;

2% myristic isopropyl;

0.2% Germall 115;

0.2% Phenova;

3% glycerin;

3% Sepigel 305;

75.1-58.6% water per 100 g of final mixture. If necessary the materials are passed through a #10-12 mesh-screen to remove aggregates. Each of the procedures should be performed with precautions against exposure to the vapors that are formed, and particularly against their inhalation. After 30 minutes of thorough mixing the formula is placed into glass jars.

This cream was tested on 62 volunteers with the above mentioned conditions. Changes in the selected variables that illustrate the effect of the above rejuvenating formula on damaged skin is summarized below:

Efficacy of Rejuvenating Cream on Sever Damaged Skin:

Treatment:

| Variables observed; | before; | after; | improvement. |
|---|---|---|---|
| Skin: | | | |
| dry | 20 | 1 | 97.5% |
| Color | 19 | 1 | 95.0% |
| Wrinkles | 40 | 26 | 35.0% |
| Elasticity improved | 40 | 39 | 97.5% |
| Decrease sensitivity to: | | | |
| environment factors | 40 | 14 | 72.0% |

As may be seen, significant benefits are provided by this rejuvenating cream to the volunteers who applied it on their skin.

Oral Product 5: each of the following ingredients is placed into a commercial mixer:

1-5 g. of bovine lactoferrin;

0.5-5 g of CLA

1-5 g. of Sea buckthorn

1-5 g. omega fatty acids

1-5 g. whey proteins

sunflower oil to make a liquid or incorporate liquid in common fillers to make a capsule.

Using an administration of Topical Product 5 twice daily and oral administration of the Oral Product 5 twice daily, a significant improvement of skin condition was noted.

### EXAMPLE 6

Revitalizing Cream.

This product consists of: bovine milk glycoprotein fraction, freeze-derived bovine whey proteins, non saturated fatty acid complex; plant derived extract containing vitamins and provitamin A and omega fatty acid complex in the same proportions as set forth in Example 5 above. The above active compounds were mixed together with: 2% cetyl alcohol; 1% vaselin; 3% glycerin; 0.5% Soya oil; 1% grape-stone oil; 2% Myristic isopropyl; 0.2% Germall 115; 0.2% Phenova; 3% Sepigel; and water per 100 g of final mixture. If necessary the materials are passed through a #10-12 mesh-screen to remove aggregates. Each of the procedures should be performed with precautions against exposure to the vapors that are formed, and particularly against their inhalation. After 30 minutes of thorough mixing the formula is placed into glass jars.

This cream is effective not only for bacterial infections on the skin assaulted by a variety of physio-chemical agents, but also for skin conditions that have deteriorated to such a point where the capillary vessels have accumulated to form “spider nets”. This revitalizing cream was self-administered by one subject with severe burn on the legs. Alleviation of pain was the major sign of the effectiveness of this cream. In later stages, this cream prevented the appearance of water blister formation that occurs after second degree burns.

In summary, one of the fundamental discoveries of this invention is the unique administration of the lactoferrin containing cream to the skin together with the oral delivery of the active ingredients in the oral form of a tablet or soft-gel. As described hereinabove, the delivery of the active ingredients by mouth is in a liquid or in the form of a capsule. The liquids used or the fillers used to make a solid are common materials employed by one skilled in the art that produce medicaments that are either liquid or solid. However, other active ingredients may be incorporated into the liquid or capsule taken orally, such as Vitamin E and illustrated as follows:

Distribution of Active Ingredients

| | Active ingredients in mg/ml or mg/100 mg | | | | | |
|---|---|---|---|---|---|---|
| Softgel tablets | Lacto | CLA | Hyppo | Omega | Whey | Vit E |
| Anti Acne (Ex1) | 25 | 0 | 0 | 0 | 0 | 0 |
| Maintenance (Ex2) | 25 | 5 | 0 | 0 | 0 | 5 |
| Mature skin (Ex3) | 25 | 5 | 20 | 0 | 0 | 5 |
| Nourishing (Ex4) | 25 | 5 | 20 | 20 | 0 | 5 |
| Rejuvenating (Ex5) | 25 | 5 | 20 | 20 | 20 | 5 |

It is understood that the material used for making the creams that are not the active ingredients may be substituted by other cream forming materials.

1. The treatment of dermatological conditions in humans comprising:
administering a material containing isolated lactoferrin topically to the skin; and
administrating, in the same time frame, isolated lactoferrin orally.

2. The treatment according to claim 1 wherein said material that is topically administered is a cream.

3. A composition that can be administered topically and orally comprising:
lactoferrin; and
a conjugated linoleic acid.

4. A composition that can be administered topically and orally comprising:
lactoferrin;
an animal or plant-derived lipid; and
oblepicha.

5. A composition that can be administered topically and orally comprising:
lactoferrin;
an animal or plant-derived lipid;
oblepicha; and
an omega fatty acid.

6. A composition that can be administered topically and orally comprising:
lactoferrin;
an animal or plant-derived lipid;
oblepicha;
an omega fatty acid; and
omega fatty acids.
